(19) United States
(12) Patent Application Publication
(10) Pub. No.: US 2026/0114753 A1
(43) Pub. Date: Apr. 30, 2026

(54) APPARATUS FOR MEASURING RESPIRATION

(71) Applicant: SB SOLUTIONS INC., Ulsan (KR)

(72) Inventor: Seungup SEO, Ulsan (KR)

(73) Assignee: SB SOLUTIONS INC., Ulsan (KR)

(21) Appl. No.: 19/432,267

(22) Filed: Dec. 24, 2025

Related U.S. Application Data
(63) Continuation of application No. PCT/KR2024/005409, filed on Apr. 22, 2024.

(30) Foreign Application Priority Data
Oct. 31, 2023 (KR) 10-2023-0148009

(51) Int. Cl. A61B 5/113; A61B 5/05; H05K 1/02; H05K 1/16

(52) U.S. Cl. CPC A61B 5/1135; A61B 5/05; H05K 1/0218; H05K 1/16; A61B 2562/166; H05K 2201/10151

(57) ABSTRACT

An apparatus for measuring respiration is disclosed. According to one embodiment, the apparatus for measuring respiration may comprise a printed circuit board (PCB) on which a measurement circuit of the apparatus for measuring respiration is formed, a sensor pattern formed on the PCB, a chip formed on the PCB, and a first ground for the measurement circuit formed on the PCB. In this case, a nominal capacity of a capacitor of the sensor pattern may be controlled according to at least one of presence or absence of a second ground for the sensor pattern and whether the first ground and the second ground are separated.

### CROSS-REFERENCE TO RELATED APPLICATION(S)

This U.S. non-provisional application is a continuation application of PCT International Application PCT/KR2024/005409, which has an international filing date of Apr. 22, 2024, now published as WO/2025/095251, which claims the priority benefit of Korean Patent Application No. 10-2023-0148009, filed on Oct. 31, 2023, in the Korean Intellectual Property Office, granted as Korean Patent No. 10-2655043 on Apr. 2, 2024, the disclosures of which are herein incorporated by reference in its entirety.

### BACKGROUND OF THE INVENTION

### Field of Invention

The following description relates to an apparatus for measuring respiration.

### Description of Related Art

With increasing interest in health, research on healthcare fields using electronic devices has been actively conducted. For example, sensors mounted on an electronic device may collect information related to the electronic device, an exterior of the electronic device, or a user, and in order for the user to check a state thereof, it is most important to continuously measure bio-signals. In this regard, as technologies for monitoring an exercise state or an abnormal state of the user are required, electronic devices providing a function of checking bio-signals of the user have been developed.

### BRIEF SUMMARY OF THE INVENTION

The present disclosure provides a respiration measuring method and an apparatus for measuring respiration, capable of continuously measuring respiration of a target object by continuously measuring changes according to respiration activity of the target object using a sensor attached to the target object.

The present disclosure also provides an apparatus for measuring respiration in which a nominal capacity of a capacitor is controlled according to presence or absence of a first ground for a sensor pattern and/or whether a first ground and a second ground for a processing circuit are separated.

The present disclosure further provides an apparatus for measuring respiration in which, in order to eliminate use of a via, a sensor pattern and a chip of a processing circuit are disposed on the same surface of a printed circuit board (PCB).

The present disclosure further provides an apparatus for measuring respiration including n or more layers including a PCB layer for a ground and a PCB layer for a power supply (Vcc) in order to reduce noise.

An apparatus for measuring respiration is provided, the apparatus comprising: a printed circuit board (PCB) on which a measurement circuit of the apparatus for measuring respiration is formed; a sensor pattern formed on the PCB; a chip formed on the PCB; and a first ground for the measurement circuit, formed on the PCB, wherein a nominal capacity of a capacitor of the sensor pattern is controlled according to at least one of presence or absence of a second ground for the sensor pattern and whether the first ground and the second ground are separated.

According to one aspect, the apparatus for measuring respiration may further comprise the second ground for the sensor pattern, wherein the first ground and the second ground are separated from each other.

According to another aspect, the sensor pattern and the chip may be formed on the same first surface of the PCB.

According to another aspect, the apparatus for measuring respiration may further comprise a plurality of conductive lines connecting the sensor pattern and the chip on the same first surface.

According to another aspect, the PCB may comprise n or more layers including at least a first PCB layer on which the first ground is formed and a second PCB layer on which a power supply circuit is formed, wherein n is a natural number equal to or greater than 2.

According to another aspect, the PCB may further comprise a third PCB layer for the second ground and a fourth PCB layer on which the sensor pattern and the chip are formed.

According to another aspect, the apparatus for measuring respiration may be attached to a target object and measure information on respiration of the target object.

According to another aspect, the sensor pattern may generate a fringing field, and the measurement circuit may comprise a circuit configured to continuously measure a change in the fringing field according to respiration activity of the target object on the basis of a change in a resonant frequency generated through an oscillator or on the basis of repetitive charging and discharging of the sensor pattern.

According to another aspect, the chip may be configured to control the measurement circuit and to provide information on the continuously measured change to an outside such that information on respiration of the target object is determinable through the continuously measured change.

According to another aspect, the sensor pattern may comprise at least two electrodes horizontally spaced apart from a surface of the target object, and the fringing field may be generated through a voltage applied to the at least two electrodes.

It is possible to provide a respiration measuring method and an apparatus for measuring respiration, capable of continuously measuring respiration of a target object by continuously measuring changes according to respiration activity of the target object using a sensor attached to the target object.

It is possible to control a nominal capacity of a capacitor according to presence or absence of a first ground for a sensor pattern of the apparatus for measuring respiration and/or whether the first ground and a second ground for a processing circuit are separated.

It is possible to eliminate use of a via through an apparatus for measuring respiration in which the sensor pattern and a chip of the processing circuit are disposed on the same surface of a printed circuit board (PCB).

It is possible to reduce noise through an apparatus for measuring respiration including n or more layers including a PCB layer for a ground and a PCB layer for a power supply (Vcc).

### BRIEF DESCRIPTION OF THE DRAWINGS

FIG. 1 is a diagram illustrating an example of a change in a sensor according to respiration activity of a target object in one embodiment of the present disclosure.

FIG. 2 is a diagram illustrating an example of an internal configuration of a respiration measuring system according to one embodiment of the present disclosure.

FIG. 3 is a flowchart illustrating an example of a respiration measuring method according to one embodiment of the present disclosure.

FIG. 4 is a diagram illustrating an example of a fringing field in one embodiment of the present disclosure.

FIG. 5 is a diagram illustrating an example of a measurement circuit unit according to one embodiment of the present disclosure.

FIG. 6 is a diagram illustrating an example of an operation of a clock counter in one embodiment of the present disclosure.

FIG. 7 is a diagram illustrating another example of the measurement circuit unit according to one embodiment of the present disclosure.

FIG. 8 is a diagram illustrating an example of an operation of an ADC in one embodiment of the present disclosure.

FIGS. 9 and 10 are each diagram illustrating examples of a schematic configuration of a measurement circuit of an apparatus for measuring respiration in one embodiment of the present disclosure.

FIG. 11 is a graph illustrating an example of measuring respiration changes according to inhalation and exhalation using an apparatus for measuring respiration according to one embodiment of the present disclosure.

FIG. 12 is a graph illustrating an example of changes in signal and noise of a signal-to-noise ratio in one embodiment of the present disclosure.

### DETAILED DESCRIPTION OF THE INVENTION

Hereinafter, embodiments will be described in detail with reference to the accompanying drawings. However, various modifications may be made to the embodiments, and the scope of the claims of the patent application is not limited or restricted by these embodiments. All modifications, equivalents, and alternatives to the embodiments should be understood to be included within the scope of the claims.

The terms used in the embodiments are used for descriptive purposes only and should not be construed as limiting. Singular expressions include plural expressions unless the context clearly indicates otherwise. In the present specification, terms such as “comprise” or “have” are intended to designate that the features, numbers, steps, operations, components, parts, or combinations thereof described in the specification exist, and should be understood as not precluding in advance the existence or possibility of addition of one or more other features, numbers, steps, operations, components, parts, or combinations thereof.

Unless otherwise defined, all terms used herein, including technical and scientific terms, have the same meaning as commonly understood by a person having ordinary skill in the art to which the embodiments pertain. Such terms as are defined in a dictionary of common use shall be interpreted as having a meaning that is consistent with the meaning they have in the context of the relevant technology and shall not be interpreted as having an ideal or excessively formal meaning unless expressly defined in the present application.

In addition, in describing the embodiments with reference to the accompanying drawings, the same components are denoted by the same reference numerals regardless of the drawing numerals, and redundant descriptions thereof will be omitted. In describing the embodiments, when it is determined that a detailed description of related known technologies may unnecessarily obscure the gist of the embodiments, such detailed description will be omitted.

In addition, in describing components of the embodiments, terms such as first, second, A, B, (a), and (b) may be used. These terms are used only for distinguishing one component from another component, and do not limit the nature, order, or sequence of the corresponding components by such terms. When a component is described as being “connected,” “coupled,” or “linked” to another component, it should be understood that the component may be directly connected or linked to the other component, or another component may be connected, coupled, or linked between the respective components.

Components included in one embodiment and components including common functions will be described using the same names in other embodiments. Unless otherwise stated, descriptions described in one embodiment may be applied to other embodiments, and detailed descriptions thereof will be omitted to the extent of overlap.

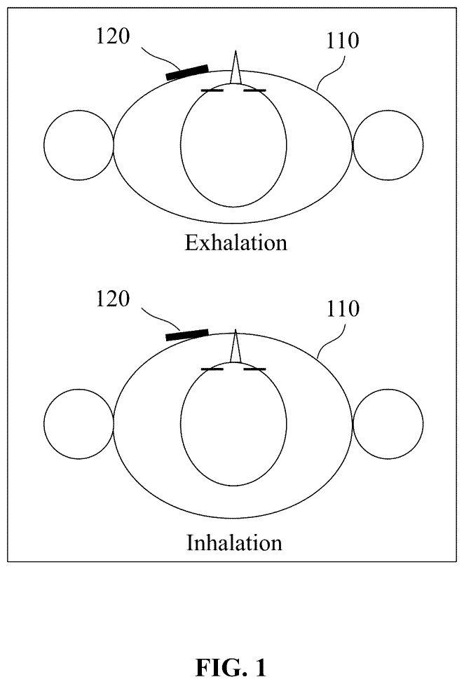

FIG. 1 is a diagram illustrating an example of a change in a sensor according to respiration activity of a target object in one embodiment of the present disclosure. FIG. 1 illustrates an example of a sensor 120 attached to a target object 110 such as a human or an animal performing respiration activity. A portion such as a thorax of the target object 110 moves while a volume thereof changes according to respiration activity of the target object 110.

The sensor 120 may be attached to such a specific portion of the target object 110. In this case, the sensor 120 may be attached such that the sensor is not completely in close contact with an external surface of the target object 110. For example, in the case of a human body, the sensor 120 may be attached such that only a part of one surface of the sensor 120 is attached to skin of the human body, such that an entire corresponding surface of the sensor 120 is not in close contact with the skin of the human body.

When the target object 110 performs respiration activity, movement occurs while the volume of the thorax changes, and according to such movement, a degree of contact between the sensor 120 and the external surface of the target object 110 continuously changes, thereby inducing a predetermined change. FIG. 1 illustrates that the degree of contact between the sensor 120 and the target object 110 differs during inhalation and exhalation of the target object 110.

A respiration measuring system according to embodiments of the present disclosure may measure information on respiration, such as a respiration pattern and or a respiration cycle of the target object 110, by continuously measuring such changes according to respiration activity of the target object 110.

In one embodiment, the respiration measuring system may form a fringing field introduced into an inside of a surface of the target object 110 by using two or more electrodes through the sensor 120. According to an embodiment, the fringing field may be formed to reach at least the surface of the target object 110. In this case, the respiration measuring system may obtain information on respiration of the target object 110 by measuring a change in the fringing field according to respiration activity of the target object 110. In this case, as a method of measuring the change in the fringing field according to respiration activity of the target object 110, an oscillator and or repetitive charging and discharging of the sensor 120 may be utilized.

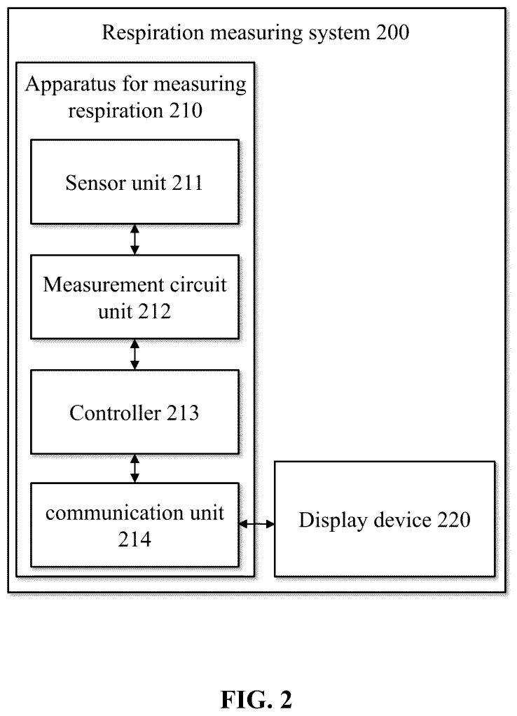

FIG. 2 is a diagram illustrating an example of an internal configuration of a respiration measuring system according to one embodiment of the present disclosure. The respiration measuring system 200 according to the embodiment of FIG. 2 may be mainly used for diagnosing whether respiration is normal and sleep apnea during sleep, and may further be used to determine respiration quality and sleep quality, but is not limited thereto.

The respiration measuring system 200 may comprise an apparatus for measuring respiration 210 and a display device 220. In the embodiment of FIG. 2, an example in which the apparatus for measuring respiration 210 and the display device 220 are implemented as separate physical devices is described. However, according to an embodiment, the apparatus for measuring respiration 210 and the display device 220 may be implemented as a single physical device.

The apparatus for measuring respiration 210 may comprise a sensor unit 211, a measurement circuit unit 212, a controller 213, and a communication unit 214.

The sensor unit 211 may be a respiration measuring sensor based on a change in a fringing field, and the measurement circuit unit 212 may comprise a measurement circuit configured to read sensor data or sensing data through the sensor unit 211. The controller 213 may control an operation of the measurement circuit unit 212 and may control the communication unit 214 to transmit the measured data to the display device 220. The communication unit 214 may comprise a communication module for a wired or wireless connection with the display device 220. Data communication between the communication unit 214 and the display device 220 may be performed using at least one of various well-known communication protocols such as Bluetooth Low Energy (BLC), Near Field Communication (NFC), and WiFi.

The display device 220 may be a user terminal such as a smartphone or a smart watch. The display device 220 may display data (for example, waveform data) measured by the apparatus for measuring respiration 210, and may display a respiration rate, respiration quality, and sleep quality of the target object 110 determined through the measured data. To this end, an algorithm for determining the respiration rate, the respiration quality, and the sleep quality may be executed in the apparatus for measuring respiration 210 or the display device 220. When the corresponding algorithm is executed in the apparatus for measuring respiration 210, the apparatus for measuring respiration 210 may further transmit, in addition to the measured data, information on the respiration rate, the respiration quality, and the sleep quality of the target object 110 determined using the measured data to the display device 220.

According to an embodiment, in order to determine sleep quality and the like, the apparatus for measuring respiration 210 may further comprise an additional sensor such as a motion sensor.

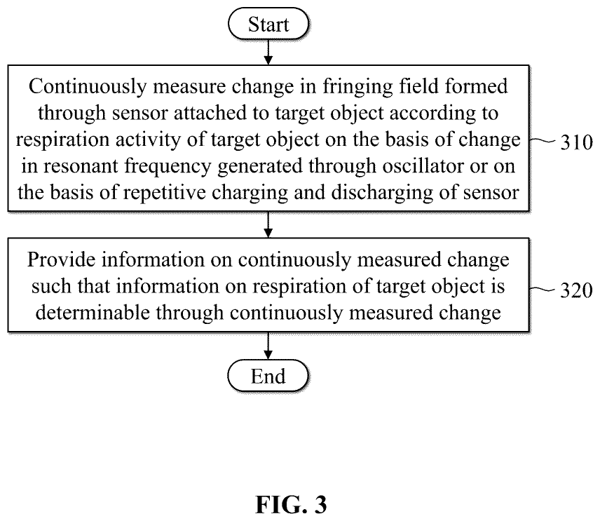

FIG. 3 is a flowchart illustrating an example of a respiration measuring method according to one embodiment of the present disclosure. The respiration measuring method according to the present embodiment may be performed by a fringing-field-based apparatus for measuring respiration 210. In one embodiment, the controller 213 of the apparatus for measuring respiration 210 may comprise at least one processor and a memory. In this case, an operation of the apparatus for measuring respiration 210 may be interpreted as being implemented as the processor of the controller 213 controls the measurement circuit unit 212 and the communication unit 214 included in the apparatus for measuring respiration 210 according to computer program code stored in the memory of the controller 213.

In step 310, the apparatus for measuring respiration 210 may continuously measure a change in a fringing field formed through a sensor attached to a target object according to respiration activity of the target object on the basis of a change in a resonant frequency generated through an oscillator or on the basis of repetitive charging and discharging of the sensor. Here, the sensor attached to the target object may correspond to the sensor 120 described above with reference to FIG. 1 or the sensor unit 211 described above with reference to FIG. 2.

The sensor may comprise at least two electrodes horizontally spaced apart from a surface of the target object. In this case, in step 310, the apparatus for measuring respiration 210 may apply a voltage to the at least two electrodes to form a fringing field. The fringing field may be formed to be introduced into an inside of the surface of the target object, or may be formed to reach at least the surface of the target object. Thereafter, the apparatus for measuring respiration 210 may measure a change in the fringing field on the basis of an oscillator or on the basis of repetitive charging and discharging of the sensor.

In one embodiment, the apparatus for measuring respiration 210 may measure a change in a resonant frequency of an oscillator according to a change in the fringing field caused by respiration activity of the target object. For example, the fringing field may be formed inside the target object or on the surface of the target object. In this case, in order to measure the change in the fringing field according to respiration activity of the target object through the change in the resonant frequency of the oscillator, the apparatus for measuring respiration 210 may count a period of an output signal of the oscillator using a clock counter and may measure a change in a counted value.

In this case, the clock counter may count periods of the output signal during a reference time generated by a reference time generator. As a frequency of the output signal increases, a relatively larger number of periods may be counted by the clock counter during the reference time.

In other words, it is possible to identify a change in the resonant frequency generated by the oscillator through a change in the counted value counted by the clock counter, which may indicate that a change in the fringing field according to respiration activity of the target object is identified. As such, by continuously measuring the change in the counted value counted by the clock counter, information on respiration of the target object may be obtained.

According to another embodiment, the apparatus for measuring respiration 210 may repeatedly charge and discharge a sensor attached to a target object. For example, the measurement circuit unit 212 included in the apparatus for measuring respiration 210 may connect and disconnect the sensor (for example a capacitive sensor) to and from a current source through a charging switch by using a control signal having a reference time interval generated through a reference time generator, thereby charging and discharging the sensor at the reference time interval. Thereafter, the apparatus for measuring respiration 210 may measure a change in a degree to which the sensor is charged according to a change in a fringing field caused by respiration activity of the target object. For example, in the apparatus for measuring respiration 210, the fringing field may change according to a change in capacitance caused by respiration activity of the target object. In this case, the change in capacitance may be measured through the change in the degree to which the sensor is charged. In this case, the measurement circuit unit 212 included in the apparatus for measuring respiration 210 may convert a voltage at an input terminal of the sensor into a digital code using an analog-to-digital converter (ADC). In this case, the measurement circuit unit 212 may measure a change in the degree to which the sensor is charged through a change in an output value of the ADC at a time point at which charging of the sensor is completed.

In other words, a change in a fringing field according to respiration activity of a target object may be reflected by a change in a degree to which the sensor is charged, and the measurement circuit unit 212 may continuously measure, each time the sensor is charged and discharged, a change in an output value at a time point at which charging of the sensor is completed (for example, a time point at which the charging switch releases a connection between the sensor and the current source). Accordingly, information on respiration of the target object may be obtained through the change in the output value of the ADC.

In step 320, the apparatus for measuring respiration 210 may provide information on a continuously measured change such that information on respiration of a target object is determinable through the continuously measured change. The information on respiration of the target object may include information on a respiration rate, respiration quality, and sleep quality described above.

In one embodiment, when the apparatus for measuring respiration 210 determines the information on respiration, the apparatus for measuring respiration 210 may provide information on the continuously measured change (namely a change in a fringing field) as an input to an algorithm driven by the controller 213. The continuously measured change may substantially correspond to a change in a resonant frequency generated through an oscillator, and the change in the resonant frequency may be obtained through a change in a counted value counted by a clock counter as described above.

In another embodiment, when information on respiration is determined by an external device of the apparatus for measuring respiration 210 such as the display device 220, the apparatus for measuring respiration 210 may provide information on the continuously measured change to the external device such as the display device 220 through the communication unit 214.

In addition, when it is determined, on the basis of information on respiration of a target object, that respiration of the target object has not occurred for a preset time or longer, a notification may be provided. For example, when the apparatus for measuring respiration 210 directly determines the information on respiration of the target object, the apparatus for measuring respiration 210 may monitor whether respiration of the target object has not occurred for the preset time or longer according to the information on respiration of the target object. In this case, the apparatus for measuring respiration 210 may provide a notification to a user through various methods such as vibration or sound. As another example, the apparatus for measuring respiration 210 may transmit a signal to the display device 220 such that the display device 220 provides a notification to the user through various methods such as vibration or sound. As another example, when the display device 220 directly determines the information on respiration of the target object, the display device 220 may monitor whether respiration of the target object has not occurred for the preset time or longer according to the information on respiration of the target object. In this case, the display device 220 may provide a notification to the user through various methods such as vibration or sound. As another example, the display device 220 may transmit a signal to the apparatus for measuring respiration 210 such that the apparatus for measuring respiration 210 provides a notification to the user through various methods such as vibration or sound. Here, the user may be the target object, a guardian of the target object, and or an administrator of the target object. In addition, the preset time may be empirically determined, for example 8 seconds or 10 seconds.

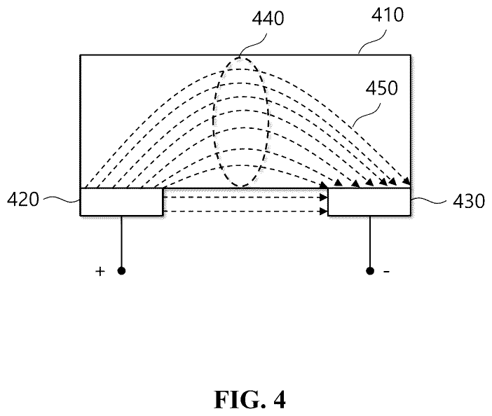

FIG. 4 is a diagram illustrating an example of a fringing field in one embodiment of the present disclosure. FIG. 4 illustrates two electrodes 420, 430 attached to a target object 410. In this case, as a voltage is applied to the two electrodes 420, 430, a fringing field 440 may be formed between the two electrodes 420, 430 and introduced into an inside of the target object 410, as illustrated in FIG. 4.

In FIG. 4, the fringing field 440 is illustrated as a dotted ellipse for convenience of description, but, in practice, the fringing field 440 may be formed by electromagnetic field lines (for example, field lines 450 illustrated in FIG. 4) between two conductors when a voltage is biased to a capacitor.

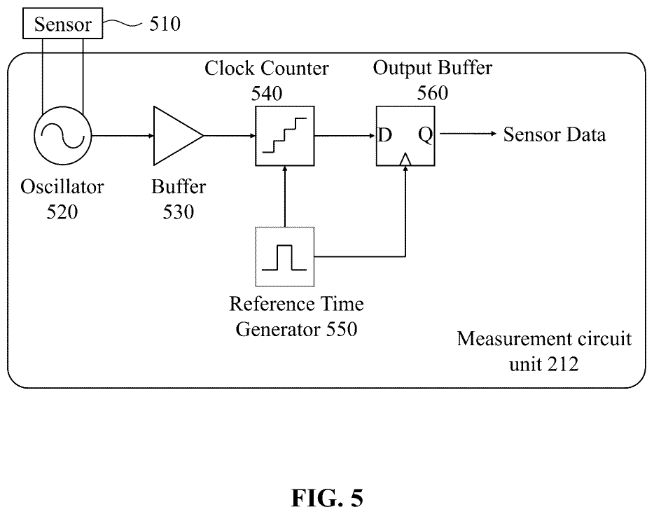

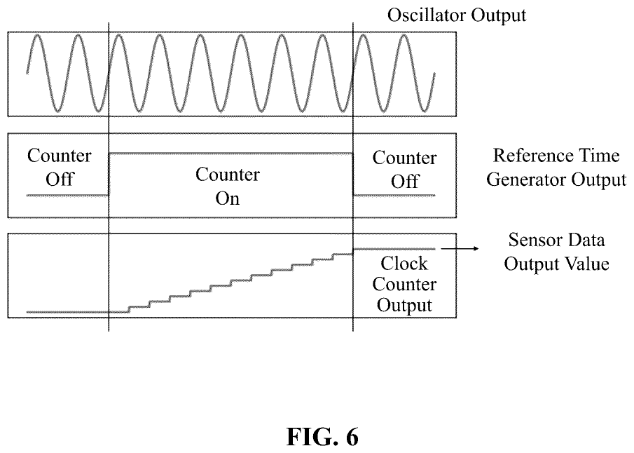

FIG. 5 is a diagram illustrating an example of a measurement circuit unit according to one embodiment of the present disclosure. FIG. 6 is a diagram illustrating an example of an operation of a clock counter in one embodiment of the present disclosure.

The measurement circuit unit 212 according to the embodiment of FIG. 5 may comprise an oscillator 520 connected to a sensor 510, a buffer 530, a clock counter 540, a reference time generator 550, and an output buffer 560.

The sensor 510 may correspond to the sensor 120 described above or the sensor unit 212, and a fringing field may be formed as a voltage is applied to at least two electrodes (for example the two electrodes 420, 430) included in the sensor 510. The oscillator 520 may be a resistor-capacitor (RC) oscillator or an inductor-capacitor (LC) oscillator. In this case, when the fringing field changes according to respiration, an output frequency (namely a resonant frequency) of the oscillator 520 connected to the sensor 510 may change. In this case, an output signal of the oscillator 520 may be input to the clock counter 540 through the buffer 530.

The clock counter 540 may count periods of an input signal during a reference time generated by the reference time generator 550. As a frequency of the input signal increases, a relatively larger number of periods may be counted during the reference time, and thus an output value of the clock counter 540 may increase. The reference time generator 550 may generate a signal of a reference time during which the clock counter 540 operates.

An output of the clock counter 540 may be output as sensor data through the output buffer 560.

FIG. 6 illustrates an example in which, when an output (which is a signal of a resonant frequency) of the oscillator 520 is input to the clock counter 540, the clock counter 540 counts periods of the output of the oscillator 520 according to an output of the reference time generator 550 and outputs a counted value as an output value of sensor data.

As such, a fringing field formed through the sensor 510 changes according to respiration of the target object 110, a resonant frequency output by the oscillator 520 changes according to the change in the fringing field, and an output value of the clock counter 540 changes according to the change in the resonant frequency. Accordingly, information on respiration of the target object 110 may be obtained through the change in the output value of the clock counter 540.

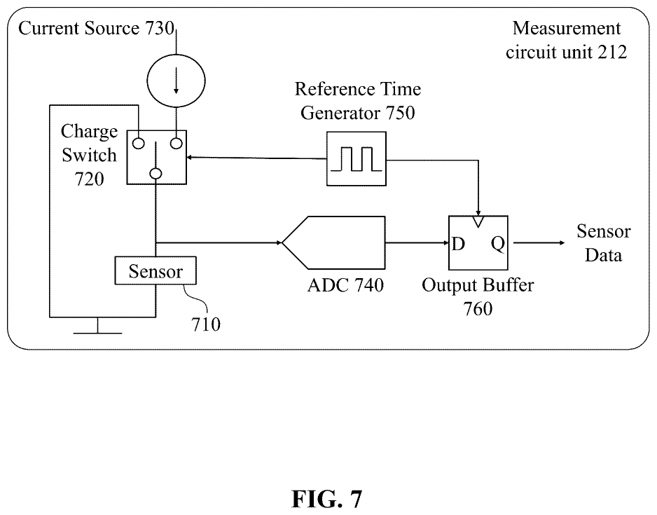

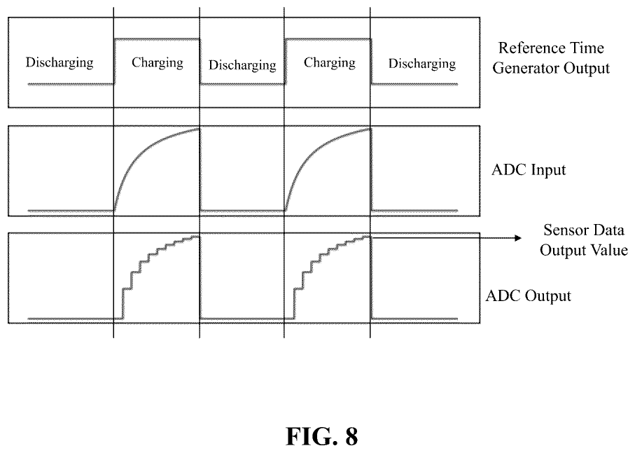

FIG. 7 is a diagram illustrating another example of a measurement circuit unit according to one embodiment of the present disclosure, and FIG. 8 is a diagram illustrating an example of an operation of an ADC in one embodiment of the present disclosure.

The measurement circuit unit 212 according to the embodiment of FIG. 7 may comprise a charge switch 720 connected to a sensor 710, a current source 730, an ADC 740, a reference time generator 750, and an output buffer 760.

The sensor 710 may correspond to the sensor 120 described above or the sensor unit 212. In the embodiment of FIG. 7, the sensor 710 is illustrated as being included in the measurement circuit unit 212, but, in practice, the sensor 710 may be disposed outside the measurement circuit unit 212 so as to be attached to the target object 110.

The measurement circuit unit 212 may repeatedly charge and discharge the sensor 710 using the charge switch 720 and may measure a degree to which the sensor is charged. The reference time generator 750 may generate a control signal having a reference time interval to operate the charge switch 720. When the charge switch 720 is turned on, the sensor 710 and the current source 730 are connected such that the sensor 710 is charged. When the charge switch 720 is turned off, the connection between the sensor 710 and the current source 730 is released such that the sensor 710 is discharged.

While the sensor 710 is being charged, a voltage at an input terminal of the sensor 710 may increase, and the measurement circuit unit 212 may convert the voltage into a digital code using the ADC 740. In this case, an output value of the ADC 740 at a time point at which the charge switch 720 is turned off by the reference time generator 750 may be output as sensor data through the output buffer 760.

FIG. 8 illustrates respective input and output of the ADC 740 according to repeated connection and disconnection between the sensor 710 and the current source 730 by the charge switch 720 in response to an output of the reference time generator 750. FIG. 8 also illustrates that an output value of the ADC 740 at a time point at which the charge switch 720 is turned off by the reference time generator 750 may be output as a sensor data output value.

As such, as a fringing field changes according to respiration of the target object 110, a degree to which the sensor 710 is charged changes, and an output value of the ADC 740 may change according to the change in the degree to which the sensor 710 is charged. Accordingly, information on respiration of the target object 110 may be obtained through a change in the output value of the ADC 740.

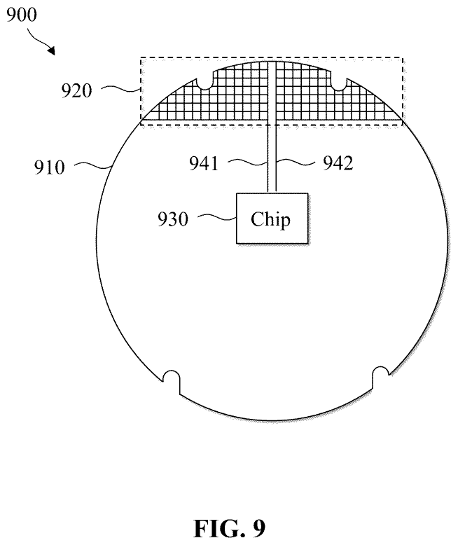

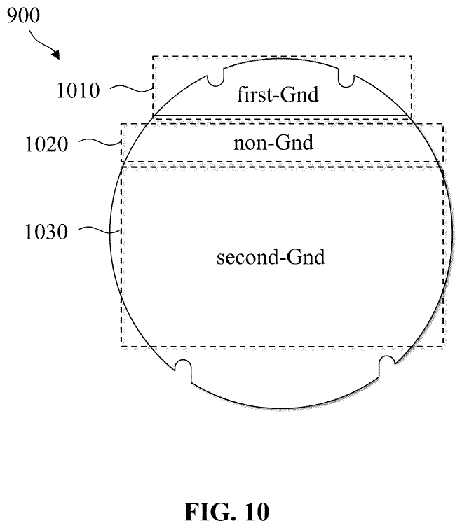

FIGS. 9 and 10 are each diagram illustrating examples of a schematic configuration of a measurement circuit of an apparatus for measuring respiration in one embodiment of the present disclosure. FIG. 9 illustrates an example briefly showing a front surface of a measurement circuit 900 of the apparatus for measuring respiration 210 described above, and illustrates a configuration in which a sensor pattern and a chip 930 are formed on a first surface (for example, a front surface) of a printed circuit board (PCB) 910. A first dashed box 920 includes a region of the PCB 910 on which the sensor pattern is formed. Here, the measurement circuit 900 may correspond to the measurement circuit unit 212 described above, the chip 930 may correspond to the controller 213, and the sensor pattern may correspond to the sensor 510, 710 described above. In FIGS. 9 and 10, illustration of remaining components other than the sensor pattern and the chip 930 of the measurement circuit 900 is omitted. In this case, the sensor pattern and the chip 930 may be connected through two conductive lines 941, 942. FIG. 10 illustrates an example briefly showing a second surface of the measurement circuit 900. A second dashed box 1010 may include a region in which a first ground (first-Gnd) for the sensor pattern is formed, a third dashed box 1020 may include a region in which no ground is formed (non-Gnd), and fourth dashed box 1030 may include a region in which a second ground (second-Gnd) for a processing circuit including the chip 930 is formed. In this case, the second ground may be necessarily included for the processing circuit, whereas the first ground may be selectively included. In addition, the first ground and the second ground may be connected as a single ground, or may be separated from each other through a region in which no ground is formed, as illustrated in FIG. 10, according to an embodiment. In this case, the apparatus for measuring respiration 210 may control a nominal capacity of a capacitor of the sensor pattern according to presence or absence of the first ground and or whether the first ground and the second ground are separated. Here, the nominal capacity may indicate capacitance of the sensor pattern and may be determined on the basis of a dielectric constant of an electrolyte and a distance between electrodes. For example, when the first ground is present and the first ground and the second ground are connected, the nominal capacity may have a value of 1 pF or less. In addition, when the first ground is present and the first ground and the second ground are separated, the nominal capacity may have a value of 2 pF or greater. Lastly, when the first ground is not present, the nominal capacity may have a value of 2 pF or less. As such, the apparatus for measuring respiration 210 may control the nominal capacity according to presence or absence of the first ground and or whether the first ground and the second ground are separated. Preferably, the apparatus for measuring respiration may be implemented to have a nominal capacity of 2 pF or greater by forming the first ground and the second ground in a separated state.

As described above, in the embodiment of FIG. 9, an example is illustrated in which the chip 930 and the sensor pattern are connected through two conductive lines 941, 942 on the same surface of the PCB 910. In the related art, as the sensor pattern is formed on a front surface of the PCB 910 and the chip 930 is formed on a rear surface of the PCB 910, a via penetrating through the PCB 910 has been required to connect the sensor pattern and the chip 930. However, since a capacitance component is also present in the chip 930, electromagnetic radiation may occur in an undesired direction, which may cause noise. Accordingly, without using a separate via, by implementing both the sensor pattern and the chip 930 on a front side of the PCB 910 and directly connecting the sensor pattern and the chip through the conductive lines 941, 942, signal attenuation and noise may be reduced.

In addition, according to an embodiment, the PCB 910 may be formed of a plurality of layers. For example, the PCB 910 may be composed of a total of four PCB layers including a top layer, a ground (Gnd) layer, a power supply (Vcc) layer, and a bottom layer. In this case, the sensor pattern, the chip 930, and the two conductive lines 941, 942 may be formed on the bottom layer, and a first ground for the sensor pattern may be formed on the top layer. A second ground for a processing circuit may be formed on the ground layer. As such, the measurement circuit of the apparatus for measuring respiration 210 may be implemented to include n or more layers including at least a PCB layer on which a ground is formed and a PCB layer on which a power supply (Vcc) circuit is formed.

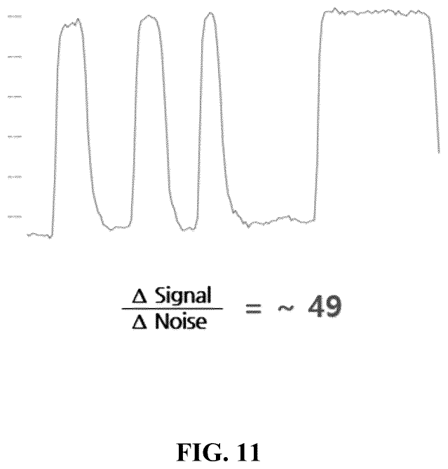

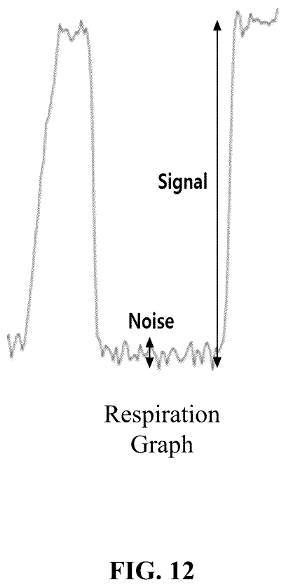

FIG. 11 is a graph illustrating an example of measuring respiration changes according to inhalation and exhalation using an apparatus for measuring respiration according to one embodiment of the present disclosure, and FIG. 12 is a graph illustrating an example of changes in signal and noise of a signal-to-noise ratio in one embodiment of the present disclosure.

As described above, according to embodiments of the present disclosure, it is possible to continuously measure respiration of a target object by continuously measuring changes according to respiration activity of the target object using a sensor attached to the target object. In addition, it is possible to control a nominal capacity of a capacitor according to presence or absence of a first ground for a sensor pattern of the apparatus for measuring respiration and or whether the first ground and a second ground for a processing circuit are separated. In addition, it is possible to eliminate use of a via through an apparatus for measuring respiration in which the sensor pattern and a chip of the processing circuit are disposed on the same surface of a PCB. Further, it is possible to reduce noise through an apparatus for measuring respiration including n or more layers including a PCB layer for a ground and a PCB layer for a power supply (Vcc).

The system or apparatus described above may be implemented by hardware components, or by a combination of hardware components and software components. For example, the apparatuses and components described in the embodiments may be implemented by using one or more general-purpose computers or special-purpose computers, such as a processor, a controller, an arithmetic logic unit (ALU), a digital signal processor (DSP), a microcomputer, a field programmable gate array (FPGA), a programmable logic unit (PLU), a microprocessor, or any other device capable of executing and responding to instructions. The processing device may execute an operating system (OS) and one or more software applications executed on the operating system. In addition, the processing device may access, store, manipulate, process, and generate data in response to execution of software. For convenience of understanding, a case in which one processing device is used has been described; however, those skilled in the art will appreciate that the processing device may include a plurality of processing elements and/or a plurality of types of processing elements. For example, the processing device may include a plurality of processors, or may include one processor and one controller. In addition, other processing configurations, such as a parallel processor, are also possible.

Software may include a computer program, code, instructions, or a combination of one or more thereof, and may configure a processing device to operate as desired or may instruct the processing device independently or collectively. Software and/or data may be embodied in any type of machine, component, physical device, virtual equipment, computer storage medium, or device in order to be interpreted by a processing device or to provide instructions or data to the processing device. Software may be distributed over network-connected computer systems and may be stored or executed in a distributed manner. Software and data may be stored in one or more computer-readable recording media.

Although the embodiments have been described above with reference to limited embodiments and drawings, those skilled in the art will appreciate that various modifications and variations are possible based on the above description. For example, the described techniques may be performed in an order different from the described order, and/or components of the described systems, structures, apparatuses, circuits, and the like may be combined or assembled in a form different from the described form, or may be replaced or substituted by other components or equivalents, while appropriate results may still be achieved.

Accordingly, other implementations, other embodiments, and equivalents to the claims also fall within the scope of the claims described below.

What is claimed is:

1. An apparatus for measuring respiration, comprising:
a printed circuit board (PCB) on which a measurement circuit of the apparatus for measuring respiration is formed;
a sensor pattern formed on the PCB;
a chip formed on the PCB; and
a first ground for the measurement circuit, formed on the PCB,
wherein a nominal capacity of a capacitor of the sensor pattern is controlled according to at least one of presence or absence of a second ground for the sensor pattern and whether the first ground and the second ground are separated,
wherein the PCB comprises n or more layers including at least a first PCB layer on which the first ground is formed and a second PCB layer on which a power supply circuit is formed,
wherein n is a natural number equal to or greater than 2, and
wherein the PCB further comprises a third PCB layer for the second ground and a fourth PCB layer on which the sensor pattern and the chip are formed.

2. The apparatus for measuring respiration according to claim 1, further comprising:
the second ground for the sensor pattern,
wherein the first ground and the second ground are separated from each other.

3. The apparatus for measuring respiration according to claim 1, wherein
the sensor pattern and the chip are formed on the same first surface of the PCB.

4. The apparatus for measuring respiration according to claim 3, further comprising:
a plurality of conductive lines connecting the sensor pattern and the chip on the same first surface.

5. The apparatus for measuring respiration according to claim 1, wherein
the apparatus for measuring respiration is attached to a target object and measures information on respiration of the target object.

6. The apparatus for measuring respiration according to claim 1, wherein
the sensor pattern generates a fringing field, and
the measurement circuit comprises a circuit configured to continuously measure a change in the fringing field according to respiration activity of the target object on the basis of a change in a resonant frequency generated through an oscillator or on the basis of repetitive charging and discharging of the sensor pattern.

7. The apparatus for measuring respiration according to claim 6, wherein
the chip is configured to control the measurement circuit and to provide information on the continuously measured change to an outside such that information on respiration of the target object is determinable through the continuously measured change.

8. The apparatus for measuring respiration according to claim 6, wherein
the sensor pattern comprises at least two electrodes horizontally spaced apart from a surface of the target object, and
the fringing field is generated through a voltage applied to the at least two electrodes.
